(19) United States
(12) Patent Application Publication
(10) Pub. No.: US 2008/0206892 A1
(43) Pub. Date: Aug. 28, 2008

(54) Rapid Magnetic Biosensor With Integrated Arrival Time Measuremnt

(75) Inventor: Menno Wille Jose Prins, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/917,215

(22) PCT Filed: Jun. 14, 2006
(86) PCT No.: PCT/IB2006/051914

§ 371 Date: Dec. 12, 2007

(30) Foreign Application Priority Data
Jun. 17, 2005 (EP) 05105359.3

(51) Int. Cl. G01N 33/553

(52) U.S. Cl. 436/526

(57) ABSTRACT

The invention provides a method, a device and a system for determining a concentration of at least one sort of polarizable or polarized magnetic labels in a fluid, the sensing surface comprising at least one sort of binding sites capable of specifically attaching to at least one sort of biological entities linked to the magnetic labels, the sensing device further comprising at least one magnetic sensor element, the sensing device further comprising first means for determining the concentration of magnetic labels attached to the binding sites and second means for determining the time of arrival of the fluid.

The present invention relates to a sensing device and to a system for determining a concentration of at least one sort of polarizable or polarized magnetic labels in a fluid, the system comprising the sensing device. The present invention further relates to a method for determining a concentration of at least one sort of polarizable or polarized magnetic labels in a fluid using the sensing device.

In the field of diagnostics especially in biomedical diagnostics, such as medical and food diagnostics for both in vivo and in vitro application, the use of biosensors or biochips is well known. These biosensors or biochips are generally used in the form of micro-arrays of biochips enabling the analysis of biological entities as e.g. DNA (desoxyribonucleic acid), RNA (ribonucleic acid), proteins or small molecules, for example hormones or drugs. Nowadays, there are many types of assays used for analysing small amounts of biological entities or biological molecules or fragments of biological entities, such as binding assays, competitive assays, displacement assays, sandwich assays or diffusion assays. The challenge in biochemical testing is presented by the low concentration of target molecules (e.g. pmol.1⁻¹ and lower) to be detected in a fluid sample with a high concentration of varying background material (e.g. mmol.1⁻¹). The targets can be biological entities like peptides, hormones, metabolites, proteins, nucleic acids, steroids, enzymes, antigens, haptens or drugs. The background material or matrix can be urine, blood, serum, saliva or other human or non-human liquids. Labels attached to the targets improve the detection limit of a target. Examples of labels are optical labels, colored beads, fluorescent chemical groups, enzymes, optical barcoding or magnetic labels.

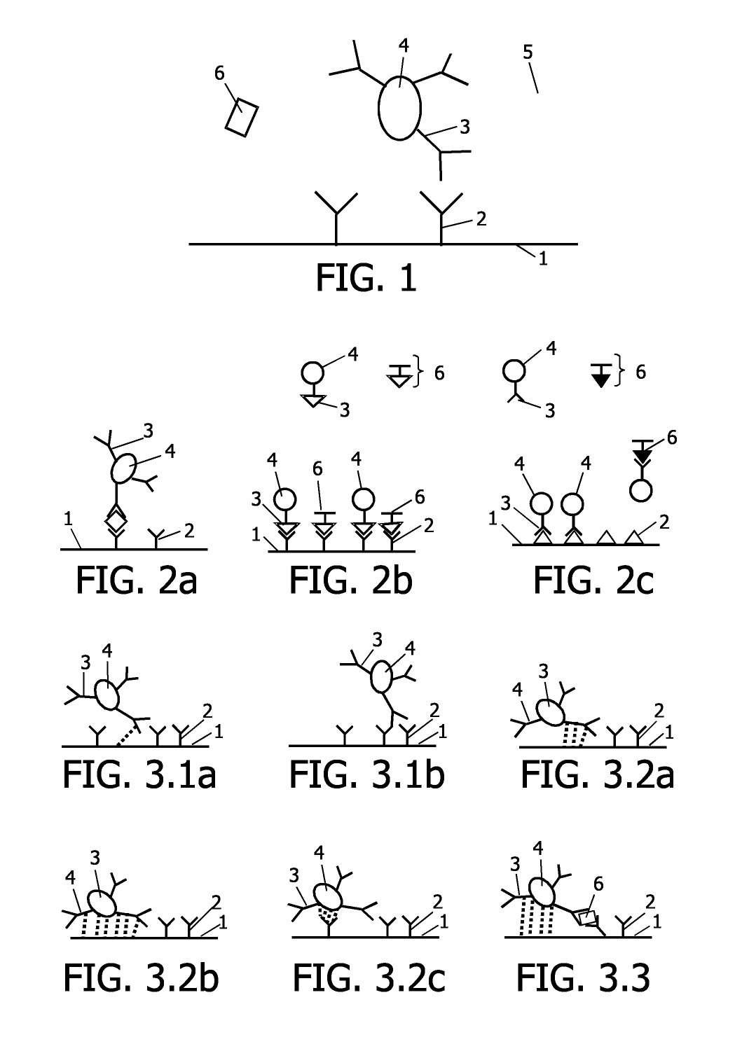

Biosensors generally employ a sensing surface 1 with specific binding sites 2 equipped with capture molecules. These capture molecules can specifically bind to other molecules or molecular complexes present in the fluid. Other capture molecules 3 and labels 4 facilitate the detection. This is illustrated in FIG. 1, which shows a biosensor sensing surface 1 to which capture molecules are coupled providing binding sites 2 to other biological entities, e.g. the target molecules 6 or targets 6. In solution 5, targets 6 and labels 4 to which further capture molecules 3 are coupled are present. Targets 6 and labels 4 are allowed to bind to the binding sites 2 of the biosensor sensing surface 1 in a specific manner which is herinafter called “specifically attached”. However, other binding configurations are possible, which are herinafter called “non-specifically attached”. In FIGS. 2a, 2b, 2c, 3.1a, 3.1b, 3.2a, 3.2b, 3.2c, 3.3 some examples of possible binding configurations of labels 4 to a biosensor sensing surface 1 are shown. FIG. 2a and 2b represent the so-called Type 1 binding configurations which realize the desired specific biological attachment. In FIG. 2a, a desired biological attachment is shown in which the target molecule 6 is sandwiched between the binding site 2 on the biosensor sensing surface 1 and a capture molecule 3 present on a label 4 (sandwich assay). In FIG. 2b, the case of a competitive assay biosensor is shown, where the binding sites 2 provided on the sensing surface 1 are able to attach both the labels 4 (by attaching the binding sites 2 to the capture molecules 3 equipped with the labels 4) and as well the targets 6. The targets 6 have, at least partially and in respect of the binding sites 2, a form and/or a behavior similar to the capture molecules 3 so that there is a competition for binding sites 2 between the capture molecules 3 (i.e. the labels 4) and the targets 6. In FIG. 2c, the case of an inhibition assay biosensor is shown, where the binding sites 2 are biologically similar to the targets 6 and where the labels 4 are bound to capture molecules 3 (or in general biological entities) that can either bind to the targets 6 or to the binding sites 2. Ideally, a target 6 bound (via a capture molecule 3) to a label 4 can no longer bind to the binding surface 2.

In contrast to this biological attachment to the sensing surface 1, labels 4 can also attach to the sensing surface 1 in a non-specific manner, i.e. bind to the surface 1 without mediation of the specific target molecules 6. FIGS. 3.1a, 3.1b, 3.2a, 3.2b, 3.2c, 3.3 represent such a non-specific attachment with FIGS. 2.2a and 2.2b showing examples of a so called Type 2 binding configuration where a single non-specific bond exists between a capture molecule 3 coupled to the label 4 and the biosensor sensing surface 1 and/or between a capture molecule 3 coupled to the label 4 and a binding site 2 coupled to the biosensor sensing surface 1. Normally, such a Type 2 binding by only a single non-specific bond is only weak and can be removed by stringency procedures such as washing or magnetic forces. As represented in FIGS. 3.2a, .3.2b and 3.2c, a so called Type 3 binding configuration to the sensing surface 1 and/or to the binding site 2 is also possible via a multitude of non-specific bonds across a larger area between the labels 4 (or the capture molecule 3 coupled to the labels 4), on the one hand, and the biosensor sensing surface 1 and/or the bindings sites 2, on the other hand. Type 3 configurations usually provide a stronger binding force than Type 1 bonds. FIG. 3.3 shows a degenerate version of Type 1, where the label 4 is bound to the biosensor sensing surface 1 by specific as well as non-specific bonds.

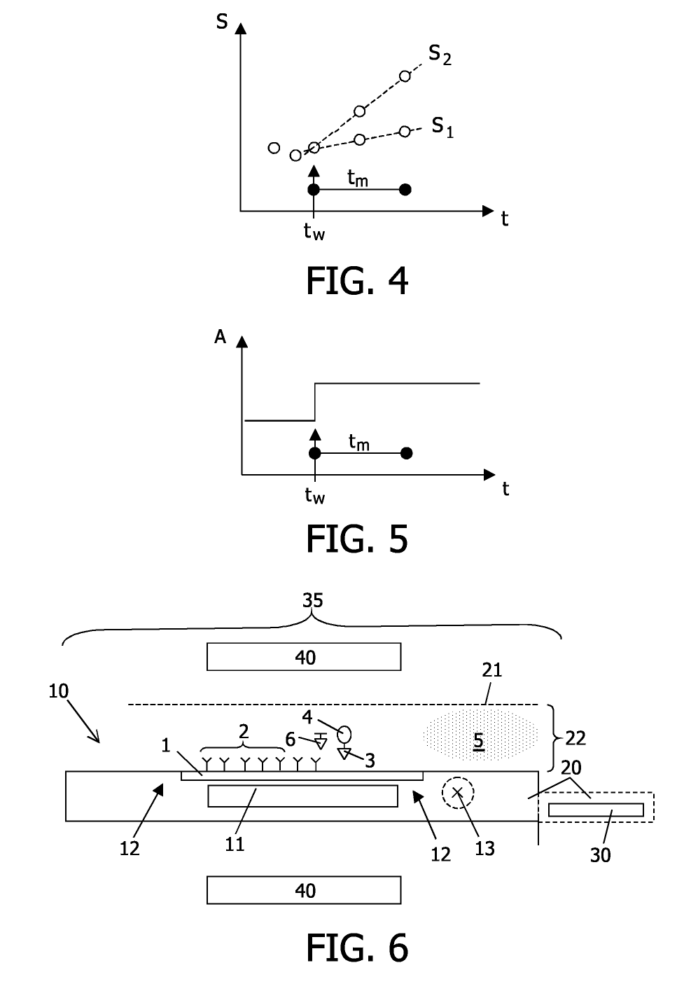

In rapid testing, e.g. roadside through-the-window testing of drugs-of-abuse in saliva, e.g. for traffic safety, it is essential to provide for test equipment sufficiently robust to be used on a day-to-day basis and to provide for a test method yielding results that are sufficiently quick and precise. Such testing can be carried out in several formats, e.g. in a competitive or in an inhibition assay format. In FIG. 4, the development over time of the target-dependent sensor signals S₁ and S₂ for two different test samples is shown, where signal S₁ corresponds to a high target 6 concentration and signal S₂ corresponds to a low target 6 concentration. The difference in S₁ versus S₂ is due to the fact that the lower the concentration of target molecules in the test sample the higher is the probability of labels 4 attached to capture molecules 3 to bind to the binding sites 2 of the sensing surface 1.

In international patent application WO 03/054566 A1, a magnetoresistive sensing device for determining a density of magnetic particles in a fluid is disclosed. The magnetoresistive sensing device or biochip has a substrate with a layer structure supporting a fluid. The layer structure has a first surface area in a first level and a second surface area in a second level and a magnetoresistive sensing element for detecting the magnetic field of at least one magnetic particle in the fluid. The magnetoresistive element is positioned near a transition between the first and the second surface area and faces at least one of the surface areas. With such a device it is possible to determine the concentration of labels 4 in the fluid.

It is an object of the present invention to provide a sensing device, a system and a method, which are able to determine a concentration of at least one sort of polarizable or polarized magnetic labels in a fluid in a manner that is sufficiently quick and accurate.

The above object is accomplished by a sensing device, a system and a method according to the present invention.

In a first aspect of the present invention, a sensing device is provided for determining a concentration of at least one sort of polarizable or polarized magnetic labels in a fluid. The sensing device comprises at least one sensing surface, the sensing surface comprises at least one sort of binding sites capable of specifically attaching to at least one sort of biological entities linked to the magnetic labels. The sensing device further comprises at least one magnetic sensor element, the sensing device further comprises first means for determining the concentration of magnetic labels attached to the binding sites and second means for determining the time of arrival of the fluid.

An advantage of the device according to the invention is that it allows to determine the concentration of the target molecules in a biological assay on a magnetic biosensor more accurately and more rapidly than previously known. It was totally surprising and could not have been expected by a person skilled in the art that it is possible to improve the detection limit and the specificity and hence reduce the time-to-result with the sensing device according to the invention by accurately determining the time of arrival of the fluid together with an accurate determination of the concentration of labels during the time that the binding process takes place on the sensing surface.

In point-of-need testing, e.g. a roadside through-the-window saliva testing of drugs-of-abuse, e.g. for traffic safety, it is essential to provide a rapid measurement with a minimal time-to-result. The time-to-result should be about a minute or, more preferred, about ten seconds. The time-to-result can be reduced in several ways, e.g. by shortening the sampling time, by enhancing the speed of the biological processes inside the disposable cartridge or by reducing the variations in the assay and improving the precision of the data. A shortening of the sampling time can be achieved, e.g. by requiring a smaller sample volume. An enhanced speed of the biological processes inside the sensor can be achieved e.g. by using biological capture molecules with a high association rate. A reduction of the variations in the assay and an improvement of the precision of the data can be achieved e.g. by having a better control of the assay, better measuring data, improved understanding of the assay and improved algorithms for extraction of the desired parameter, e.g. concentration of a drug or of a protein.

In order to meet these needs, especially an extraction of the target concentration as rapid and as accurate as possible in only a very short measurement time tₘ, it is proposed according to the invention to improve the reliability of the data produced by the sensing device, especially in the vicinity of the time of arrival t_{w} of the fluid on the sensing surface of the sensing device.

According to the invention, it is therefore proposed to integrate a first and a second means in the sensing device. The first means is very sensitive to the concentration of target molecules in the sample. The second means is very sensitive to the presence of the fluid on the sensing surface and further, the second means is essentially insensitive to the target concentration in the sample.

According to one embodiment of the invention, the first and second means can be represented by one and the same structure on the sensing device, e.g. a two-dimensional wire structure applying a resulting magnetic field to the magnetic labels in the fluid above the sensing surface. In this embodiment it is possible to control the structure such that a precise measurement of both the target concentration and the time of arrival of the liquid is possible. This can be achieved either by measuring simultaneously or successively the target concentrations of labels above the sensing surface and the time of arrival by means of a measurement of the label concentration in the bulk of the fluid.

In general, a sensing device will be sensitive to labels specifically attached to the sensing surface (Type 1 binding, cf. above) as well as to labels that are not specifically attached but still in the vicinity of the sensing surface. This second alternative can be realized either by label binding to the sensing surface in the manner of Type 2 or by labels not attached to the sensing surface, but being located in the vicinity of the surface. According to the present invention, the first means is able to measure these different magnetic label concentrations independently, e.g. differentiate the specifically attached magnetic labels from other labels through their differences of rotational and/or translational mobility of labels specifically attached versus labels non-specifically attached. For example, it is possible to apply magnetic fields and to determine mobility-dependent signals. Such magnetic fields can also be modulated, e.g. by current wires or magnets, to attract magnetic labels to the sensing surface, or to repel magnetic labels from the sensing surface, or to move magnetic labels over the sensing surface. A comparison of the signal of the magnetic sensor element for the different positions of the magnetic labels allows a determination of the number of mobile magnetic labels in the vicinity of the sensing surface that are present in the solution to be measured. According to the invention, it is preferred to provide the first means such or to control the first means such that a time-resolved measurement of the different magnetic label concentrations is possible. In this context, the wording “time-resolved” means that it is possible to monitor or measure the build-up of the target-dependent signal S of the first means during the measuring inverval. According to an alternative of the invention, it is also possible to measure the target-dependent signal S of the first means only at the end of the measuring time.

In addition to a measurement of the concentration of magnetic labels in the vicinity of the sensing surface, i.e. a measurement of the target concentration in the sample, the present invention proposes the integration of a possibility to measure the presence or absence of fluid in the sensing device above the sensing surface.

In a preferred embodiment of the present invention, the second means is a capacitive sensing means. Such a capacitive sensing means can be realized e.g. by way of a capacitor-like structure in the sensing device. The fluid above the sensor changes the dielectric constant of the medium above the sensor, which can be accurately measured by two electrical conductors with a capacitive coupling, e.g. in the form of two wires or two capacitor plates.

In a further preferred embodiment of the present invention, the second means is a thermal sensing means. The presence of fluid changes the thermal conditions (temperature, thermal conduction), which can be measured, e.g. by a temperature-dependent resistor on the sensing device.

In still a further preferred embodiment of the present invention, the second means is a magnetic sensing means. The presence of magnetic particles, e.g. magnetic labels or other magnetic material, dispersed in the bulk of the fluid can generate a magnetic signal. Such a signal is not specific of the target concentration in the fluid as the total label concentration in the bulk of the liquid is given by reagents supplied to the sample independently of the target concentration. The magnetic particles or labels in the bulk of the fluid can e.g. be detected via their static or dynamic magnetic moment, or via translational or rotational mobility upon application of magnetic fields. In a preferred embodiment of the present invention with the second means being magnetic sensing means, magnetic field generating means may be provided on the structure of the sensing device. These magnetic field generating means may for example be a current wire or a two-dimensional wire structure. The magnetic field generating means may generate a rotating magnetic field. In another embodiment, the magnetic field generating means may generate a unidirectional or one dimensional magnetic field, e.g. a pulsed unidirectional magnetic field, or a sinusoidally modulated magnetic field. In this case, where the second means is a magnetic sensing means, the different motional or rotational freedom of magnetic labels in the bulk of the liquid above the sensing surface or bound to the binding sites of the sensing surface may be related to these different label concentrations.

In a further preferred embodiment of the present invention, the first means of the sensing device comprise two magnetic field generating means positioned on each side of one magnetic sensor element, i.e. left and right or above and below. Alternatively, the sensor element is positioned in between two current lines, e.g. parallel current sheets. An advantage of this kind of embodiments of the present invention is that the magnetic sensor element is partially or completely insensitive to the magnetic field of the two magnetic field generating means, provided that the two magnetic fields compensate each other at the location of the sensor element. Therefore, the magnetic sensor element only feels the magnetic field due to the presence of magnetic labels on the sensing surface or in the vicinity of the sensing surface. By placing the magnetic sensor element in a volume where the net magnetic field to which the sensor element is sensible is compensated by the two magnetic field generating means, possible saturation of the sensor element is avoided.

In a further preferred embodiment of the present invention, the magnetic field generating means is a two-dimensional wire structure located on the sensing device.

For all embodiments of the sensing device, the magnetic sensor element may be one of an AMR, a GMR or a TMR sensor element. Of course, magnetic sensor elements based on other principles like Hall sensor elements or SQUIDs are also possible according to the present invention.

In the following, the present invention will mainly be described with reference to magnetic labels, also called magnetic beads or beads. The magnetic labels need not necessarily be spherical in shape, but may be of any suitable shape, e.g. in the form of spheres, cylinders or rods, cubes, ovals etc. or may have no defined or constant shape. By the term “magnetic labels”, is understood that the labels include any suitable form of one magnetic particle or more magnetic particles, e.g. magnetic, diamagnetic, paramagnetic, superparamagnetic, ferromagnetic, that is any form of magnetism which generates a magnetic dipole in a magnetic field, either permanently of temporarily. For implementing the present invention, there is no limitation as to the shape of the magnetic labels, but spherical labels are at present the easiest and cheapest to manufacture in a reliable way. The size of the magnetic labels is not per se a limiting factor of the present invention. However, for detecting interactions on a biosensor, small sized magnetic labels will be advantageous. When micrometer-sized magnetic beads are used as magnetic labels, they limit the downscaling because every label occupies an area of at least 1 μm². Furthermore, small magnetic labels have better diffusion properties and generally show a lower tendency to sedimentation than large magnetic beads. According to the present invention, magnetic labels are used in the size range between 1 and 3000 nm, more preferably between 5 and 500 nm.

In the present description and claims of the invention, the term “biological entities” should be interpreted broadly. It includes bioactive molecules such as proteins, peptides, RNA, DNA, lipids, phospholipids, carbohydrates like sugars, or similar. The term “biological entities” also includes cell fragments such as portions of cell membranes, particularly portions of cell membranes which may contain a receptor. The term biological entities also relates to small compounds which potentially can bind to a biological entity. Examples are hormones, drugs, ligands, antagonists, inhibitors and modulators. The biological entities can be isolated or synthesized molecules. Synthesized molecules can include non-naturally occuring compounds such as modified aminoacids or nucleotides. The biological entities can also occur in a medium or fluid such as blood or serum or saliva or other body fluids or secretions, or any other sample comprising biological entities such as food, water samples or other.

The present invention also includes a system for determining a concentration of at least one sort of polarizable or polarized magnetic labels in a fluid, the system comprising a sensing device according to any one of the previously described embodiments. The system comprises the sensing device together with a suitable mechanical environment such as packages, chambers, channels, tubes or the like for sampling, sample pretreatment, wetting the sensing surface, etc. The system further comprises the sensing device together with suitable electrical and/or electronical environment such as power supply, data collection and analysing means, output means.

The present invention also includes a method for determining a concentration of at least one sort of polarizable or polarized magnetic labels in a fluid using the sensing device according to any one of the previously described embodiments of the sensing device, the method comprising the steps of:
providing a fluid comprising magnetic labels over the sensing surface,
determining the time of arrival of the fluid,
determining the concentration of magnetic labels attached to the binding sites.

These and other characteristics, features and advantages of the present invention will become apparent from the following detailed description, taken in conjunction with the accompanying drawings, which illustrate, by way of example, the principles of the invention. The description is given by way of example only, without limiting the scope of the invention. The reference figures quoted below refer to the attached drawings.

FIG. 1 illustrates a biosensor to which binding sites are coupled in a solution comprising targets and labels to which capture molecules are coupled.

FIGS. 2a, 2b, 2c, 3.1a, 3.1b, 3.2a, 3.2b, 3.2c, 3.3 illustrate some examples of possible binding configurations of labels 4 to a biosensor sensing surface.

FIG. 4 illustrates the time development of sensor signals for two different test samples of high target concentration and low target concentration.

FIG. 5 illustrates the time development of the signal of the second means according to the present invention for a time interval where an arrival of fluid on the sensing device occurs.

FIG. 6 illustrates a schematic representation of a system and a sensing device according to the present invention.

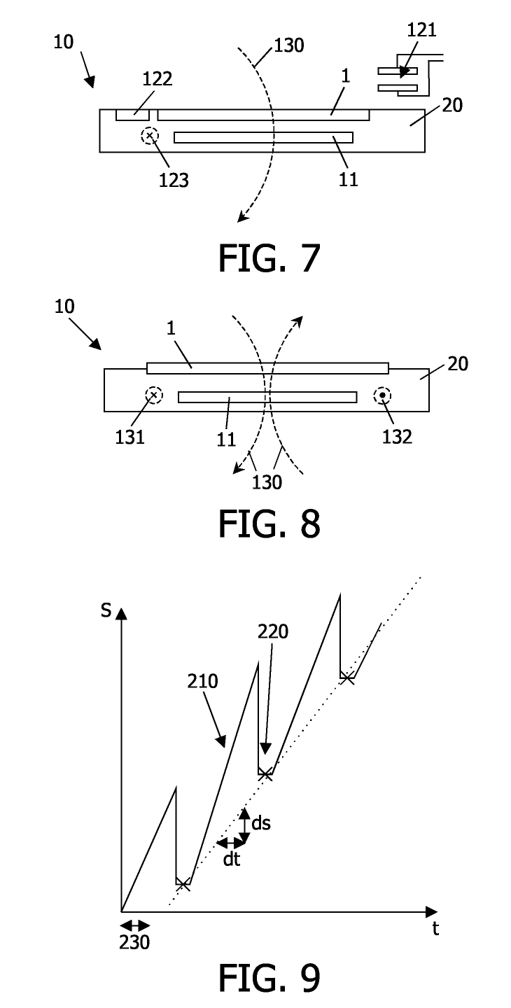

FIG. 7 illustrates a schematic representation of a sensing device with different possibilities of the second means.

FIG. 8 illustrates a schematic representation of a device according to the further embodiment of the present invention.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn to scale for illustrative purposes.

Where an indefinite or definite article is used when referring to a singular noun, e.g. “a”, “an”, “the”, this includes a plural of that noun unless otherwise specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices constisting only of components A and B. It means that with respect to the present invention, the only relevant components of the device are A and B.

In FIGS. 1 to 3 have already been described in the introductory part of the description.

In FIG. 4, the development over time of the target-dependent sensor signals S₁ and S₂ for two different test samples is shown. The signal strength depends on the target concentration, in a way that depends on the type of assay. In the example of a competitive-type or an inhibition-type assay, signal S₁ corresponds to a high target concentration and signal S₂ corresponds to a low target concentration. In the examples of a sandwich assay, an anti-complex assay, or a selective antibody assay with blocking agent, the reverse applies, i.e. signal S₁ corresponds to a lower target concentration than signal S₂. Over a time interval tₘ, which corresponds to the measuring time, it is possible to measure the target concentrations with sufficient accuracy. Several small circles in FIG. 4 denote measurements actually performed by the sensing device. The time interval tₘ corresponds to the time-to-result of the sensing device. At the beginning of the measuring time interval is the time of arrival t_{w} of the fluid, especially a liquid, above the sensing surface. According to the present invention, the moment t_{w} is measured with high precision by means of the second means. Note that the figure gives an example of a more or less linear behavior of the signal versus time. In some cases the signal can be more complicated, e.g. like a higher order polynome, due to for example an activation time of the biological layer, or a diffusion time or drift time of beads toward the sensor surface.

In a magnetic biosensor, the measurement of the concentration of specifically bound beads (or labels) to the surface can be perturbed by the presence of unbound or non-specifically bound beads (or labels). Therefore, a reliable data point for measuring a target concentration through a label concentration is best taken when unbound and non-specifically bound beads are removed from the surface.

Therefore, the following sequence or cycle can be applied in a repeated manner:
pull the beads towards the surface; thereby binding may take place
then the beads must be pulled from the surface, to distinguish between specific binding to the surface and non-specific binding or unbound beads.
after this magnetic ‘washing’ step one can measure the actual signal.

FIG. 9 sketches the surface-sensitive signal that can be expected as a function of time and how the slope of the surface-binding curve can be derived. In the context of the present invention, the surface-sensitive signal is also called the raw signal of the target-dependent sensor signal S (or S₁, S₂ depicted in FIG. 4). The above-mentioned sequence or cycle is used to determine the slope of the signal represented by the dotted line. The signal represented by the dotted line is identical to the target-dependent sensor signal S. Therefore, the measured slope of this signal leads to a determination of the target concentration in the fluid sample. The sequence or cycle mentioned above is also represented in FIG. 9, where reference sign 210 denotes the step of pulling labels to the surface and where reference sign 220 denotes the step of pulling labels from the surface. Reference sign 230 denotes a single sample interval or a “unitary measurement” event in the manner of the small circles in FIG. 4. During the measuring time tₘ (cf FIG. 4), a certain number of such sample intervals need to be collected.

The slope of the curve is proportional to the binding rate of labels to the sensor surface. The average slope dS/dt of the signal during the measuring time tₘ is given by the signal S (at the end of tₘ) devided by the measuring time tₘ. The target concentration is related to the binding rate, in a fashion that depends on the assay. The target concentration can be very accurately determined when the signal is recorded with a high signal-to-noise ratio. In the case of detection by a magneto-resisitve biosensor, a high signal-to-noise ratio can be achieved by the use of high currents. High currents can cause heating or irreversibly change bio-materials. However, when the signal is measured at the endpoint of the assay, heating and changes of the biomaterials are not important. In other words, the endpoint signal can be measured with a very high signal-to-noise ratio, which enhances the precision of a determination of the target concentration.

FIG. 5 shows a diagram of the output signal A of the second means 12. At the time t_{w}, the output signal A varies strongly such that an electronic circuit, preferably integrated into the substrate of the sensing device, can accurately deduce the time of arrival t_{w}, which is also called the wetting time, of the fluid on the sensing surface.

In FIG. 6, a system 35 and a sensing device 10 are shown. The present invention provides a sensing device 10 such as, e.g. a biosensor or a biochip, especially suitable for use as a biosensor-array, i.e. a multitude of biosensors arranged on one single substrate material. The sensor or chip substrate can be any suitable mechanical carrier, of organic or inorganic material, e.g. glass, plastic, silicon, or a combination of these. The sensing device 10 is part of a system 35 according to the present invention. In a preferred application of the sensing device 10 of the present invention, the sensing device 10 is used in a test kit for road-side through-the-window testing of drugs-of-abuse in saliva for traffic safety. By way of example, this device is equipped for a competitive assay (cf. FIG. 2b). The sensing device 10 comprises a sensing surface 1 where binding sites 2 are located. The binding sites 2 are provided to specifically bind to capture molecules 3 and targets 6. The targets 6 are biological entities (e.g. drugs of abuse) and the capture molecules 3 are target-like molecules that have been coupled to the labels 4. Entities 3 and 6 can both bind to sites 2, therefore this is called a competitive assay format. The device can also be equipped for an inhibition assay (cf. FIG. 2c) but for the sake of simplicity, only the case of a competitive assay is explained in this paragraph. The sensing device 10 comprises a substrate 20. It is preferred but not mandatory that the sensing device 10 comprises magnetic field generating means integrated into the first means 13. At least if no magnetic field generating means are provided in the substrate 20 of the sensing device 10, a magnetic field generating device 40 external to the sensing device 10 is usually present in the inventive system 35. In FIG. 5 is illustrated an embodiment of the system 35 comprising both internal magnetic field generating means located in or on the substrate 20 of the sensing device 10 and magnetic field generating means 40 external to the sensing device 10. The system 35 further comprises a housing 21 forming at least a channel 22 or chamber 22 or the like for providing sufficient space for the fluid 5, especially a liquid, containing the target-like capture molecules 3 attached to the labels 4. Furthermore, the fluid 5 comprises the targets 6.

In another preferred embodiment, the device of FIG. 6 is equipped for an inhibition assay format (cf. FIG. 2c). In that case the binding sites 2 are target-like molecules coupled to the sensor surface 1. The targets 6 are biological entities such as drugs of abuse or the like and the capture molecules 3 are biological entities (e.g. anti-target antibodies) that can specifically bind to targets 6 and target-like binding sites 2. This is called an inhibition assay format because the binding of targets 6 to labels 4 partially or totally inhibits the binding of label 4 to the target-like binding sites 2.

As is clear from the above two examples, the device can be equipped for a range of different assay formats, e.g. competitive, inhibition, displacement, sandwich assay. As is known in the art, the biochemical and chemical species (e.g. targets, target-like molecules, labels, binding sites) can be brought together at once or sequentially. For enhanced speed, it is advantageous to bring the reagents together at once. In the latter case, the kinetics of the processes and the factual sequence of binding processes depends e.g. on the diffusion and binding rates.

In a preferred embodiment of the sensing device 10, an electronic circuit 30 is provided in the substrate 20. The electronic circuit 30 is provided to collect signals or data collected or measured by a magnetic sensor element 11 located in the substrate 20. In an alternative embodiment of the present invention, the electronic circuit 30 can also be located outside the substrate 20. The electronic circuit 30 also processes the output signal A produced or induced by the second means 12, which in FIG. 6 are only illustrated by arrows 12.

The magnetic field generating means as an example for the first means 13 may, for example, be magnetic materials (rotating or non-rotating) and/or conductors such as, e.g. current wires. In the embodiment described, the magnetic field generating means is preferably generated by means of current wires. Detection of rotational and/or translational movement of labels 4 may preferably be performed magnetically. The magnetic detection may preferably be performed by using the integrated magnetic sensor element 11. Various types of sensor elements 11 may be used such as, e.g. a Hall sensor, magneto-impedance, SQUID, or any of the suitable magnetic sensor. The magnetic sensor element 11 is preferably provided as a magnetoresistive element, for example, a GMR or a TMR or an AMR sensor element 11. A rotating magnetic field generating means can be provided by means of current wires as well as current generating means integrated in the substrate 20 of the sensing device 10. The magnetic sensor element 11 may have, e.g., an elongated (long and narrow) strip geometry. The rotating magnetic field is thus applied to the magnetic labels 4 by means of current flowing in the integrated current wires. Preferably, the current wires may be positioned in such a way that they generate magnetic fields in the volume where magnetic labels 4 are present.

In FIG. 7, a schematic representation of a sensing device 10 with different possibilities of the second means 12 is shown. On or in the substrate 20 of the sensing device 10 is provided the sensing surface 1 and the sensor element 11. For the sake of simplicity, the first means are not shown in FIG. 7 but only three different embodiments 121, 122, 123 of the second means 12. In the first embodiment, the second means 12 is provided as a capacitive sensing means 121. In the second embodiment, the second means 12 is provided as a thermal sensing means 122. In the third embodiment, the second means 12 is provided as a magnetic sensing means 123. According to a further embodiment of the invention, the sensing device 10 can also comprise the second means 12 in the form of two or more structures of a capacitive sensing means 121 and/or a thermal sensing means 122 and/or a magnetic sensing means 123.

A capacitive sensing means 121 can be provided by means of two electrodes, e.g. capacitor plates or wires, on or above the sensing surface 1 or in the vicinity of the sensing surface 1. The capacitor plates can be provided as metallized areas on or nearby the sensing surface 1. Alternatively, the capacitor plates can be provided in the form of areas of a semiconductor material such as silicon, polysilicon or any other suitable material. The capacitor plates can be arranged substantially parallel to the plane of the substrate 20, which is positioned substantially opposite one another in a direction normal to the plane of the substrate 20. This has the advantage that an important sample volume is covered or taken into account for the measurement of the time of arrival t_{w}. Alternatively, the capacitor plates can be positioned substantially opposite one another in a direction parallel to the plane of the substrate 20. This has the advantage, that the capacitor plates can be manufactured substantially in the same plane as the sensing surface 1, which reduces the complexity of the manufacturing process of the sensing device 10.

A thermal sensing means 122 can be provided in the form of an electrical impedance working as a temperature sensor. It is generally known in the art to provide layers of resistive materials on semiconductor devices or other substrates, the materials showing a relatively large temperature coefficient regarding electrical conductivity. Such materials include metals such as platinum, aluminum, copper, gold, alloys or other materials such as amorphous or polycrystalline semiconductors. Alternatively, dielectrical materials can be used, measuring the temperature via a change of dielectric properties. The thermal sensing means 122 can be realized according to the invention, e.g. as a structured layer of a material with electrical impedance, for example in a meander-like structure in or on the substrate 20 of the sensing device.

For both the capacitive sensing means 121 as well as the thermal sensing means 122, it is possible to provide at least two structures of the sensing means 121, 122 in the direction of flow of the fluid entering the sensing device before and after the sensing surface 1 or at the beginning and at the end of the sensing surface 1. By that, it is possible to measure the time t_{w} still with higher precision because it is possible to deduce from the signals produced or induced by the two structures of capacitive and/or thermals sensing means 121, 122 an accurate estimate of the actual time of arrival of the fluid at the location of the sensing surface 1, i.e. at the location where the biological processes take place, even in the case where the fluid arrives with low speed at the sensing device.

A magnetic sensing means 123 can be provided in the form of a wire structure in or on the substrate 20 producing a magnetic field at a location where the bulk concentration of magnetic labels 4 is accessible. The magnetic sensing means 123 can be integrated into the first means 13 if the first means 13 comprise a magnetic field generating means. Alternatively, the magnetic sensing means 123 can be separated from the first means 13. For example, according to the invention, it is possible that the magnetic sensing means 123 comprises not only a magnetic field generating means (integrated or separated from a magnetic field generating means of the first means) but also a further sensor element distinct from the sensor element 11 integrated into the inventive sensing device 10. By that provision it is possible to independently and simultaneously measure the time of arrival t_{w} and the target-dependent signal S by different sensor elements. According to an alternative embodiment of the present invention, both measurements can also be effected by one and the same sensor element or sensor elements. Using the same electrical elements, different signals can be extracted, for example by using time-modulated currents or voltages. When a magnetic sensor is electrically excited at frequency fi and a neighbouring current wire at frequency f₂, signals at f₁ and f₂ have contributions due to capacitive coupling, while signals at f₁±f₂ relate to magnetic cross-talk, e.g. due to the presence of magnetic particles.

FIG. 8 shows a schematic representation of a sensing device 10 according to a further embodiment of the present invention. In the substrate 20 is located the sensing surface 1 and the magnetic sensor element 11. Furthermore, a first magnetic field generating means 131 and a second magnetic field generating means 132 are located in the substrate 20 of the sensing element 10 creating together magnetic field 130. It can be seen in FIG. 8 that at the location of the magnetic sensor element 11, the components of the magnetic fields created by the first and second magnetic field generating means 131, 132 compensate, at least in respect of a component of the resulting magnetic field to which the magnetic sensor element 11 is sensitive.

The applied magnetic field 130 is such that it generates a torque on the labels 4. In that way, the labels 4 are rotated with respect to another body (e.g. another label 4, the sensing surface 1, etc) using the magnetic field 130. As previously stated, the labels 4 contain a magnetic material known in the art. The label 4 may, for example, be a magnetic bead, a magnetic particle, a magnetic rod, a string of magnetic particles or a magnetic material inside a non-magnetic matrix. A parameter relating to the rotational or motional freedom of the labels 4 can be detected by the sensing device 10. The method according to the invention allows high-frequency motional freedom or rotational freedom measurements. By way of measurements of this kind, a distinction between the magnetic label concentration in the bulk of the fluid 5 versus the magnetic label concentration in the vicinity of the sensing surface is possible.

The precise knowledge of the time of arrival t_{w} can also be used for other control purposes of the biosensor. For example, it is possible that the sensing device according to the present invention runs in a sensitivity-gauge or calibration mode before the time of arrival t_{w} and in a sensitive-detection mode after t_{w}. By this provision, it is possible that the sensing device according to the present invention produces more accurate results. Another possibility of the use of the precise knowledge of the time of arrival t_{w} is that the currents can be kept low, e.g. to save power: after t_{w}, the electrical currents should be increased to achieve the highest possible measurement sensitivity. Furthermore, the precise knowledge of t_{w} can be used to optimize the thermal conditions of the sensing surface 1: before t_{w} the thermal conditions are different than after tw, e.g. because of the heat conductivity of the fluid sample. In order to have optimal thermal conditions, useful for the stability of the biological layer on the sensing surface and for optimal conditions for rapid and specific biological binding, the optimal heating by current wires shall be different before and after t_{w}.

Preferably, the second means—which is sensitive to the presence of fluid on the sensing surface—is also very sensitive to the presence of a gas bubble. A gas bubble can perturb the biological assay, so the detection of presence or absence of a gas bubble is an important check for the reliability of the sensor data.

We note that the above invention can be combined with sensor multiplexing and/or label multiplexing. In sensor multiplexing, sensors are used with different types of binding sites 2. Also the capture molecules 3 on the labels 4 can be of different types. In label multiplexing, different types of labels 4 are used, e.g. labels with different sizes or different magnetic properties.

1. A sensing device (10) for determining a concentration of at least one sort of polarizable or polarized magnetic labels (4) in a fluid (5),
the sensing device (10) comprising at least one sensing surface (1),
the sensing surface (1) comprising at least one sort of binding sites (2) capable to specifically attaching to at least one sort of biological entities (3) linked to the magnetic labels (4)
the sensing device (10) further comprising at least one magnetic sensor element (11),
the sensing device (10) further comprising first means (13) for determining the concentration of magnetic labels (4) attached to the binding sites (2) and second means (12) for determining the time of arrival of the fluid.

2. A sensing device (10) according to claim 1, wherein the second means (12) is a capacitive sensing means.

3. A sensing device (10) according to claim 1, wherein the second means (12) is a thermal sensing means.

4. A sensing device (10) according to claim 1, wherein the second means (12) is a magnetic sensing means.

5. A sensing device (10) according to claim 1, wherein the first means (13) comprise magnetic field generating means (13) for generating a magnetic field (130).

6. A sensing device (10) according to claim 5, wherein the first means (13) comprise two magnetic field generating means (131, 132) positioned on each side of one magnetic sensor element (11).

7. A sensing device (10) according to claim 5, wherein the magnetic field generating means (13) is a two-dimensional wire structure located on the sensing device (10).

8. A sensing device (10) according to claim 5, wherein the magnetic field generating means (13) generates a rotating magnetic field (130).

9. A sensing device (10) according to claim 5, wherein the magnetic field generating means (13) generates a unidirectional magnetic field (130).

10. A sensing device (10) according to claim 1, wherein the magnetic sensor element (11) is a magnetoresistive sensor element, preferably an AMR, a GMR or a TMR sensor element.

11. A sensing device (10) according to claim 1, wherein the magnetic labels (4) are provided as magnetic beads.

12. A system (35) for determining a concentration of at least one sort of polarizable or polarized magnetic labels (4) in a fluid (5), the system (35) comprising the magnetoresistive sensing device (10) according to claim 1.

13. A system (35) according to claim 12, further comprising an electronic circuit (30) for detecting a change in magnetoresistance of the magnetic sensor element (11), the electronic circuit (30) being present in the substrate (20).

14. A system (35) according to claim 12, further comprising external magnetic field generating means (40) for generating a magnetic field.

15. A method for determining a concentration of at least one sort of polarizable or polarized magnetic labels (4) in a fluid (5) using the sensing device (10) of claim 1, the method comprising the steps of:
providing a fluid (5) comprising magnetic labels (4) over the sensing surface (1),
determining the time of arrival of the fluid,
determining the concentration of magnetic labels (4) attached to the binding sites (2).

16. A method according to claim 15, wherein the magnetic determining of the time of arrival of the fluid and the determining of the concentration of magnetic labels (4) attached to the binding sites (2) is performed using the difference of rotational and/or translational mobility of labels (4) in the bulk of the fluid versus labels (4) attached to the binding sites (2).
